(19) United States
(12) Patent Application Publication
(10) Pub. No.: US 2013/0190594 A1
(43) Pub. Date: Jul. 25, 2013

(54) Scanning Optoacoustic Imaging System with High Resolution and Improved Signal Collection Efficiency

(71) Applicants: Alexander A. Oraevsky, Houston, TX (US); Dmitri Tsyboulski, Houston, TX (US); Andre Conjusteau, Houston, TX (US)

(72) Inventors: Alexander A. Oraevsky, Houston, TX (US); Dmitri Tsyboulski, Houston, TX (US); Andre Conjusteau, Houston, TX (US)

(21) Appl. No.: 13/748,315

(22) Filed: Jan. 23, 2013

Related U.S. Application Data
(60) Provisional application No. 61/632,388, filed on Jan. 23, 2012.

(51) Int. Cl. A61B 5/00; A61B 5/145

(52) U.S. Cl. CPC A61B 5/0095; A61B 5/72; A61B 5/14542; A61B 5/14546
USPC 600/407

(57) ABSTRACT

Provided herein are scanning, high-resolution optoacoustic imaging systems or microscopes. Generally, the system/microscope comprises subsystems for scanning a tissue or object therein with a wavelength of electromagnetic energy, such as optical energy, collecting and detecting ultrasonic waves produced when the tissue or object absorbs the incident wavelength and converting the same to an electrical signal, and for processing, analyzing and displaying the electrical signal as a digital image. Specifically, the system/microscope utilizes an off-axis parabolic reflector with a high numerical aperture value for deep tissue visualization. Also, provided is a method for collecting volumetric image data voxel-by-voxel within a subject utilizing the imaging system or microscope. A series of voxels within the scanned tissue produces detectable ultrasonic waves that are collected by the off-axis parabolic reflector and processed as described as a high-resolution image of the tissue or object therein.

### CROSS-REFERENCE TO RELATED APPLICATION

This non-provisional application claims benefit of priority under 35 U.S.C. §119(e) of provisional application U.S. Ser. No. 61/632,388, filed Jan. 23, 2012, now abandoned, the entirety of which is hereby incorporated by reference.

### BACKGROUND OF THE INVENTION

b 1. Field of the Invention

The present invention generally relates to the fields of optoacoustic methods and apparatus suitable for imaging purposes, and in particular, but not limited to, to microscopy, endoscopy and ophthalmoscopy systems dedicated to tissue imaging, monitoring, and sensing to provide quantitative feedback on molecular features and vasculature.

2. Description of the Related Art

Current systems and methods of microscopic analysis in clinical studies based on high resolution optoacoustic imaging are limited in their capability to provide sufficient sensitivity and contrast at depths exceeding 1-2 mm, even though functional information from microvasculature regarding the total hemoglobin concentration and the level of blood oxygen saturation is critically important through the entire depth of tissue, such as retina, skin or a wall of an internal hollow organ. As a result, current approaches frequently provide insufficient clinically relevant information.

Optoacoustic tomography is an imaging technique that is emerging as a powerful tool for diagnostic biomedical imaging. Relying on the photoacoustic effect, where local absorption of laser light by tissues induces sound to be released, optoacoustic tomography can be used with in vivo and in vitro samples, requiring little to no sample preparation. Within clinical applications, near-infrared light can specifically target blood for imaging of vascular structures and blood distribution and to determine oxygenation and perfusion information directly based on the varying absorbances of oxygenated blood versus deoxygenated blood. Also, quantitative optoacoustic imaging with exogenous contrast agents enable imaging with molecular specificity.

Optoacoustic microscopy is a version of the optoacoustic imaging that achieves higher resolution below 100 micrometers by utilizing shorter ultrasound wavelengths and ultrasound focusing elements with larger apertures. It is well understood by those skilled in imaging sciences that theoretical resolution limit is defined by the imaging wavelength and the aperture of the ultrasound collection. One must either decrease the imaging wavelength or increase collection aperture to achieve better resolution. Conventional scanning optoacoustic microscopes consist of a laser light delivery system to generate ultrasound upon tissue illumination, an optical assembly to direct light onto tissue, and a focused sensor to detect the resulting ultrasound which impacts a transducer. An electrical signal in response to pressure is generated by the transducer which can be converted into a digital signal for interpretration and reconstruction of an image.

The prior scanning optoacoustic microscopy designs differ primarily in their method of collecting and detecting the spherical ultrasound wavefront emitted from a single point. In one example, a focused transducer that preferably has an ideal spherical surface is utilized for ultrasound detection. Due to technological limitations and difficulties in making high-frequency spherically focused transducers with frequencies up to 20 MHz and higher and dimensions of several millimeters and higher these designs provide limited resolution due to their low numerical aperture (NA). In another example, acoustic lenses made of highly refractive materials for ultrasound, such as fused silica, are employed to transform a spherical wavefront into a plane wave and to direct it to a flat transducer. The disadvantages of these setups include high losses of ultrasound energy at the refractive element that may reach up to 90%, as well as wavefront distortion due to ultrasound refraction into shear wave and longitudinal wave components inside the acoustic lens, especially at larger apertures. As a result, optoacoustic and acoustic microscopes based on these designs having refractive elements on the scale of 5-10 mm feature relatively low numerical apertures of about 0.2-0.4.

The highest numerical aperture known in the prior art is 0.74 for an acoustic microscope, but only for microscopic lens with an extremely small radius of 15 micrometers, which is not useful for tissue imaging. Lower NA values of an acoustic lens requires the use of high frequencies of ˜50 MHz or higher for tissue imaging with resolution below 100 μm which severely limits imaging depth to approximately 1 mm. The strong attenuation of high frequency ultrasonic waves in tissue makes contrast lower and deep (>1-2 mm) tissue imaging impossible. In addition, even the present optoacoustic microscopy systems utilize resonant (reverberating) transducers that detect relatively narrow band of frequencies from inherently broadband optoacoustic signals generated in tissues, which results in significant losses in sensitivity and decrease in axial resolution by a factor of ˜3 as compared to ultrawide-band (non-reverberating) ultrasonic transducers.

Thus, there is a present need for optoacoustic imaging systems with improved capabilities in deep tissue visualization. Specifically, the prior art is deficient in an optoacoustic imaging system or microscope utilizing an off-axis parabolic reflector with a high numerical aperture value for deep tissue visualization with high contrast and resolution and for providing quantitative feedback on blood distribution, tissue oxygenation, blood perfusion and tissue molecular composition. The present invention fulfills this longstanding need and desire in the art.

### SUMMARY OF THE INVENTION

The present invention is directed to a scanning three dimensional optoacoustic imaging system. The system comprises a plurality of subsystems. An electromagnetic energy delivery subsystem has a source of electronmagnetic energy deliverable to an object of interest in a subject. An ultrasound collection subsystem comprises an off-axis parabolic reflector disposed in a focusable relationship with a voxel of the object of interest that received the electromagnetic energy where the voxel generates a detectable ultrasonic waves. An ultrasound detection subsystem comprises at least one ultrawide-band ultrasonic transducer configured to convert the detectable ultrasonic waves to an electrical signal. A scanning subsystem is disposed in movable relationship to the ultrasound collection subsystem and ultrasound detection subsystem. An electronic subsystem comprises analog amplification, analog-to-digital conversion and digital signal processing components configured for acquisition and digital conversion of the electrical signals. A computer, comprising a memory and a processor, tangibly stores software configured for signal processing, image processing and reconstruction where the computer is in electronic communication with the electronic subsystem and the electromagnetic energy delivery subsystem.

The present invention is directed to a related optoacoustic imaging system further comprising a housing subsystem. An enclosure encloses the ultrasound collection subsystem and the ultrasound detection system. A coupling medium fills the enclosure. A membrane that is transparent to both electromagnetic radiation and ultrasound disposed within the enclosure and containing the coupling medium therein.

The present invention is directed to another related optoacoustic imaging system further comprising a laser ultrasound imaging system for dual modality optoacoustic/laser ultrasound imaging where laser ultrasound imaging system configured to operate with the subsystems described herein. The laser ultrasound imaging system has an absorbing layer that generates a broadband ultrasound pulse upon illumination with an optical pulse and means for directing optical pulse onto the said absorbing layer.

The present invention also is directed to a scanning optoacoustic microscope. The optoacoustic microscope comprises a source of a single or of multiple optical wavelengths absorbable by an object of interest. An off-axis parabolic reflector having a high numerical aperture is movably disposed in a focusing relationship onto a voxel within the object of interest. An ultrawide-band ultrasonic transducer array is disposed in a movable relationship with the off-axis parabolic reflector where the transducer array detects a range of ultrasonic wavelengths convertible to an electrical signal. A scanning assembly is disposed in a movable relationship to both the off-axis parabolic reflector and the transducer array. A computer system is in electronic communication with the laser and with electrical signal acquisition and transmission components where the computer system, comprising a memory, a processor and a display, tangibly stores software configured for signal processing and image reconstruction and processing.

The present invention is directed to a related optoacoustic microscope. The optoacoustic microscope further comprises a housing enclosing the off-axis parabolic reflector and the ultrawide-band ultrasonic transducer array. An optoacoustic coupling medium fills the housing and an optically and acoustically transparent membrane, disposed within the housing, contains the coupling medium therein.

The present invention is directed to another related optoacoustic imaging system further comprising a laser ultrasound imaging system for dual modality imaging with the optoacoustic microscope.

The present invention is directed further to a scanning three-dimensional optoacoustic imaging method for collecting volumetric image data voxel-by-voxel in a subject. The method comprises scanning a tissue of interest in the subject with optical energy that has a wavelength absorbed by one or more voxels within the tissue or a molecule therein. Ultrasonic waves that are generated by each voxel within the tissue or molecule are collected as each absorbs the optical energy via the off-axis parabolic reflector comprising the scanning optoacoustic microscope described herein. The collected ultrasonic waves are detected with the ultrawide-band ultrasonic transducer array comprising the scanning optoacoustic microscope and the wavefront distortions therein are corrected. The corrected ultrasonic waves are converted to electrical signals and are processed to a digital signal format. The digital signals are transmitted to the computer system for reconstruction as a volumetric image of the optical energy absorbed by each of the voxels.

The present invention is directed to a related method further comprising converting the volumetric image of the absorbed optical energy into functional and molecular images of the tissue or molecule therein.

The present invention is directed to another related method further comprising combining the optoacoustic microscope in dual modality with a laser ultrasound imaging system to acquire and co-register anatomical images of tissue morphology with the functional and molecular images.

Other and further aspects, features, and advantages of the present invention will be apparent from the following description of the presently preferred embodiments of the invention given for the purpose of disclosure.

### BRIEF DESCRIPTIONS OF THE DRAWINGS

So that the matter in which the above-recited features, advantages and objects of the invention, as well as others that will become clear, are attained and can be understood in detail, more particular descriptions of the invention briefly summarized above may be had by reference to certain embodiments thereof that are illustrated in the appended drawings. These drawings form a part of the specification. It is to be noted, however, that the appended drawings illustrate preferred embodiments of the invention and therefore are not to be considered limiting in their scope.

FIG. 1 depicts the scanning high-resolution optoacoustic/ultrasonic imaging system with high efficiency of signal collection.

FIG. 2 is an interior view of the main imaging module of the high-resolution optoacoustic imaging system.

FIGS. 3A-3B are a volumetric rendering of an ultrasound wavefront (FIG. 3A) and a cross-section of the reflector (FIG. 3B).

FIGS. 4A-4B illustrate a cross-section of an off-axis parabolic reflector (FIG. 4A) and the change of Y across the reflector surface (FIG. 4B).

FIG. 5 is a polar plot of variations in numerical aperture values of the off-axis parabolic reflector.

FIGS. 6A-6B illustrate frequency response (FIG. 6A) and frequency spectrum (FIG. 6B) of the ultrawide-band ultrasonic transducer used in the imaging system.

FIGS. 7A-7D are temporal profiles of impulse response signals from an optically-absorbing microsphere in a phantom.

FIGS. 8A-8D are maximum intensity projection images of a single optically absorbing microsphere with diameter of ˜35 micrometers contained in a phantom (FIGS. 8A, 8C) and the cross-sections through the images (FIGS. 8B, 8D).

FIGS. 9A-9B are optoacoustic images (FIG. 9A) of the diffraction limited point spread functions reconstructed from signals (FIG. 9B) with a temporal of a Gaussian derivative.

### DETAILED DESCRIPTION OF THE INVENTION

As used herein in the specification, “a” or “an” may mean one or more. As used herein in the claim(s), when used in conjunction with the word “comprising”, the words “a” or “an” may mean one or more than one.

As used herein “another” or “other” may mean at least a second or more of the same or different claim element or components thereof. Similarly, the word “or” is intended to include “and” unless the context clearly indicates otherwise. “Comprise” means “include.”

As used herein, the term “about” refers to a numeric value, including, for example, whole numbers, fractions, and percentages, whether or not explicitly indicated. The term “about” generally refers to a range of numerical values (e.g., +/−5-10% of the recited value) that one of ordinary skill in the art would consider equivalent to the recited value (e.g., having the same function or result). In some instances, the term “about” may include numerical values that are rounded to the nearest significant figure.

As used herein, the term “optoacoustic imaging” refers to imaging based on optically induced acoustic signals and also is referred to as photoacoustic imaging or thermoacoustic imaging.

As used herein, the term “parabolic reflector” referes to an acoustic reflector with a surface that can be generated by a rotation of a parbola about its axis.

As used herein, the term “aperture” defines an angle between any direction coming from a focal point of the system and the imaging central axis passing through a focal point of the system.

As used herein, the term “numerical aperture” defines the sinus of maximum angle received by the detection system of the device in any given half plane defined by the imaging central axis passing through a focal point of the system.

As used herein, the term “computer” or “computer system” refers to any networkable tabletop or handheld electronic device comprising a memory, a processor, a display and at least one wired or wireless network connection. As is known in the art, the processor is configured to execute instructions comprising any software programs or applications or processes tangibly stored in computer memory or tangibly stored in any known computer-readable medium.

As used herein, the term “subject” refers to a human or other mammal or animal or to any portion or body part thereof on which any optoacoustic imaging, as described herein, may be performed.

In one embodiment of the present invention there is provided a scanning three dimensional imaging system, comprising a) an electromagnetic energy delivery subsystem having a source of electronmagnetic energy deliverable to an object of interest; b) an ultrasound collection subsystem comprising an off-axis parabolic reflector of ultrasound with its focal volume or voxel positioned inside the object of interest, said voxel absorbing the electromagnetic energy and generating the detectable ultrasonic waves; c) an ultrasound detection subsystem comprising at least one ultrawide-band ultrasonic transducer configured to convert the detectable ultrasonic waves to an electrical signal; d) a scanning subsystem disposed in movable relationship to the ultrasound collection subsystem and ultrasound detection subsystem, whereas said scanning subsystem may provide translational and rotational types of motions; e) an electronic subsystem comprising analog amplification, analog-to-digital conversion and digital signal processing components configured for acquisition and digital conversion of the electrical signals; and f) a computer comprising a memory and a processor tangibly storing software configured for signal processing, image processing and reconstruction, said computer in electronic communication with the electronic subsystem and the electromagnetic energy delivery subsystem.

Further to this embodiment the optoacoustic imaging system may include a housing subsystem that has an enclosure enclosing the ultrasound collection subsystem and the ultrasound detection subsystem; an acoustically transparent coupling medium which is also transparent to the electromagnetic energy directed onto the object of interest, where the coupling medium fills the enclosure; and an acoustically transparent membrane which is also transparent to the electromagnetic energy directed onto the object of interest, where the membrane is disposed within the enclosure and contains the coupling medium therein.

In another further embodiment the optoacoustic imaging system comprises a laser ultrasound imaging system for dual modality optoacoustic/laser ultrasound imaging, said laser ultrasound imaging system configured to operate with the subsystems described supra and having a) an absorbing layer that generates a broadband ultrasound pulse upon illumination with an optical pulse, and b) means for directing optical pulse onto the said absorbing layer.

In one aspect of these embodiments the source of electromagnetic energy may be a laser configured to pulse a specific wavelength of optical energy. In another aspect the source of electromagnetic energy is a physical source configured to pulse or to produce a modulated continuous wave of a wavelength in a range of about 100 nm to about 10 cm.

In all embodiments and aspects the numerical aperture of the off-axis parabolic reflector may appear in a range of about 0.40 to about 0.99. Also, the at least one ultrawide-band ultrasonic transducer may have a bandwidth in a range of about 1MHz to about 50 MHz. Particularly, the at least one ultrasonic transducer may comprise a transducer array. Examples of an ultrawide-band ultrasonic transducer are a piezoelectric transducer, an optical transducer or a capacitive micromachined transducer. In addition the computer is electronically connected to a means for displaying the reconstructed image. Furthermore the object of interest may be a tissue or particles or molecule contained therein.

In another embodiment of the present invention there is provided a scanning optoacoustic microscope, comprising a source of a single or multiple optical wavelengths absorbable by an object of interest; an off-axis parabolic reflector having a high numerical aperture movably disposed in a focusing relationship onto a voxel within the object of interest; an ultrawide-band ultrasonic transducer array disposed in a movable relationship with the off-axis parabolic reflector, said transducer array detecting a range of ultrasonic wavelengths convertible to an electrical signal; a scanning assembly disposed in a movable relationship to both the off-axis parabolic reflector and the transducer array; an electronic subsystem comprising analog amplification, analog-to-digital conversion and digital signal processing components configured for acquisition and digital conversion of the electrical signals; and a computer system in electronic communication with the laser and with electrical signal acquisition and transmission components, said computer system, comprising a memory, a processor and a display, tangibly storing software configured for signal processing and image reconstruction and postprocessing.

Further to this embodiment the optoacoustic microscope may comprise a housing enclosing the off-axis parabolic reflector and the ultrawide-band ultrasonic transducer array; an optoacoustic coupling medium filling the housing; and an optically and acoustically transparent membrane made of flexible plastic or similar material disposed within the housing and containing the coupling medium therein.

In another further embodiment of the scanning optoacoustic microscope the system comprises an laser ultrasound imaging subsystem comprising a) an absorbing layer that generates a broadband ultrasound wavefront upon illumination with an optical pulse, said wavefront directed at the object under examination, b) means for directing optical pulse onto the said absorbing layer.

In all embodiments the source of a single or multiple optical wavelengths may comprise one or more lasers configured to produce pulses of optical energy at a selected wavelength or wavelengths. Also, the frequency range of the detectable ultrasonic wavelengths may appear in the range of about 1 MHz to about 50 MHz. In addition the numerical aperture of an off-axis parabolic reflector may be about 0.4 to about 0.99. Furthermore, the object is a tissue, or particles or molecules contained therein.

In yet another embodiment of the present invention there is provided a scanning three-dimensional optoacoustic imaging method for collecting volumetric image data voxel-by-voxel in a subject, comprising the steps of a) scanning a tissue of interest in the subject with optical energy having a wavelength absorbed by one or more voxels within the tissue or a molecule therein; b) collecting ultrasonic waves generated by each voxel within the tissue or molecule as each absorbs the optical energy via the off-axis parabolic reflector comprising the scanning optoacoustic microscope described supra; c) detecting the collected ultrasonic waves with the ultrawide-band ultrasonic transducer array comprising the optoacoustic microscope and correcting wavefront distortions therein; and d) converting the corrected ultrasonic waves to electrical signals which are processed to a digital format and transmitted to the computer system for reconstruction as a volumetric image of the optical energy absorbed by each of the voxels.

Further to this embodiment, the method comprises converting the volumetric image of the absorbed optical energy into functional and molecular images of the tissue or molecule therein. In another further embodiment, the method comprises combining the optoacoustic microscope in dual modality with a laser ultrasound imaging system to acquire and co-register anatomical images of tissue morphology with the functional and molecular images.

In all embodiments, the scanning step may comprise delivering the optical energy through an optoacoustic coupling medium contained within an optically and acoustically transparent membrane. Also, in all embodiments the collecting step may comprise positioning the off-axis parabolic reflector confocally with the optical energy on the tissue of interest such that each voxel in the tissue of interest is located at the common focal point on the same axis.

In addition, the off-axis parabolic reflector may convert spherical acoustic waves generated by each voxel that are incident at an ultrawide range of angles into a planar acoustic wave propagated as ultrasound. Furthermore, the corrected electrical signal may corresponds to an acoustic planar wavefront. Further still, the tissue may be vascular tissue and the molecule may be one or both of hemoglobin or oxygen.

Provided herein are systems and methods for scanning three-dimensional optoacoustic imaging, such as an optoacoustic microscopy system, that uses an off-axis parabolic reflector which has advantages over current scanning optoacoustic microscopy systems. The imaging system described herein enables an induction of optoacoustic responses from blood or other absorbers in tissue by illuminating the tissue with at least one input pulse of laser light. The transient ultrasound waves generated from the tissue are collected selectively from the focal area inside the tissue with specially designed off-axis parabolic reflector. Unlike refractive elements typically employed nowadays in acoustic and optoacoustic microscopes, the parabolic surface, made of materials with a sufficiently high shear wave velocity, will work as an ideal reflector of ultrasound which will convert an incoming spherical wavefront into a planar wave without any loss at the surface of the reflector in a widest possible range of apertures.

This conversion ability enables the use of readily available flat-surface transducers or transducer arrays with any bandwidth for detecting full frequency spectrum of the optically generated ultrasound signals. Increased aperture will improve its sensitivity and imaging depth in tissues by allowing the use of lower frequency limits for imaging to achieve the desired resolution as compared to prior optoacoustic microscopy designs. Ultra wideband detection will further further improve image contrast and allow obtaining quantitative functional and molecular information from acquired optoacoustic images.

It is advantageous to select a coupling medium to specifically match speed of sound in tissue or object under investigation to avoid bluring of the system's focus with depth. It is also advantageous to employ an array of transducers for ultrasound detection to enable dynamic corrections of distorted ultrasound wavefront, by introducing appropriate time delays into each channel in images postprocessing to ensure signals coming from the same point will effectively reach each transducer channel simultaneously. Furthermore, a transducer array enables dynamic focusing which allows a focal area or voxel imaged during a single scanning step to be expanded, thereby reducing the time needed to scan the entire volume. Thus, the present invention provides a method and apparatus for backward-mode optoacoustic microscopy with highest possible numerical aperture up to date that can achieve ultimate resolution limit defined by diffraction and provide most sensitive detection of ultrasound emitted from tissue.

Generally, the scanning three-dimensional imaging system comprises means for delivering optical energy to a volume of interest or molecules therein within a subject, means for detecting ultrasonic waves and means for processing the detected ultrasonic waves, including means for displaying the processed ultrasonic waves as an image. Such means embody various electronically interconnected subsystems for operation of thereof. Moreover the imaging system may be combined with an ultrasound imaging system, known in the art, to operate in dual modality. This configuration enables acquisition and coregistration of anatomical images of tissue morphology with functional and molecular images.

A first energy delivery subsystem comprises a source of optical energy that is configured for delivery thereof at a wavelength specific for absorption by an object of interest contained within a volume of tissue. Absorption results in the generation of acoustic waves that are propagated from the irradiated volume as ultrawide-band ultrasound. The optical energy source may be a laser source of pulsed laser energy or other physical source of electromagnetic energy with a wavelength in the range from 10 nm to 10 cm. The electromagnetic energy be pulsed or a modulated continuous wave.

A second ultrasound collection subsystem is configured for detecting only the ultrasonic waves generated in the focal area of the system by using the specially designed off-axis parabolic reflector described herein. The off-axis parabolic reflector made of material with sufficiently high shear wave velocity to convert spherical acoustic waves into a planar acoustic wave without any loss at the surface of the reflector. The off-axis parbolic reflector may have a numerical aperture of about 0.4 to about 0.99. The collection subsystem may be positioned so that it is confocal with the optical energy so that each voxel of interest is located at the common focal point on the same axis.

Particularly, the ultrasound collection subsystem comprises an off-axis parabolic reflector, preferably a 90 degrees off-axis parabolic reflector, with high numerical aperture greater then 0.4, whereas said reflector may or may not represent an ideal parabolic surface. In addition, said off-axis parabolic reflector will comprise a material with sufficiently high shear wave velocity, in order to provide a complete reflection of ultrasound coming from its focus in preferably every point on the surface of the reflector via the effect of total internal reflection.

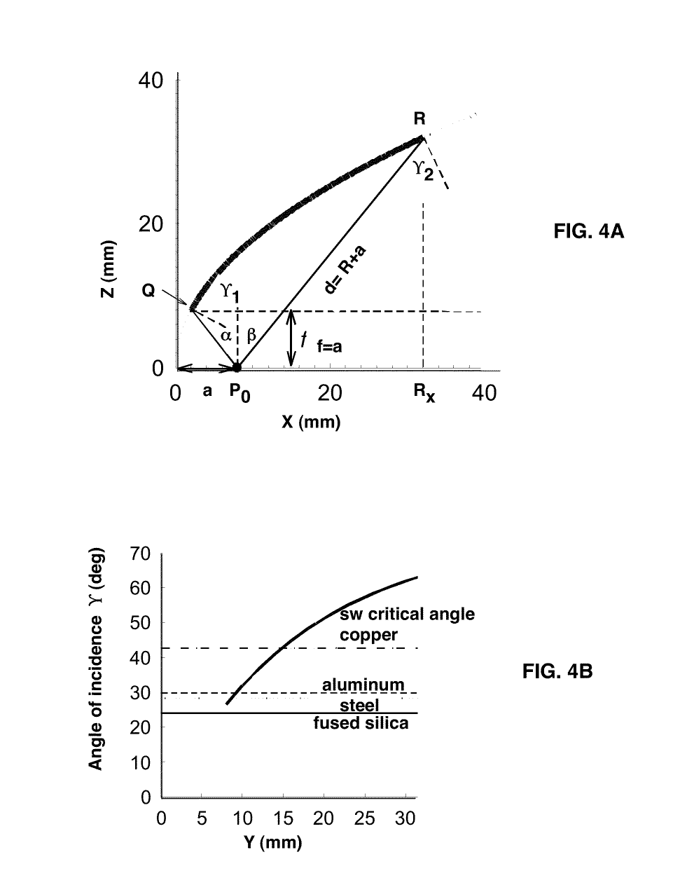

Depending on the selection of maximum imaging depth, defined by parameter f (see FIGS. 4A-4B), numerical apertures of an off-axis parabolic reflector may appear in the range 0.4-0.99.

A third ultrasound detection subsystem contains at least one ultrawide-band ultrasonic transducer or a two-dimensional array of ultrasonic transducers with a bandwidth that are sensitive to ultrasonic waves within frequency range from about 1 MHz to 50 MHz, which include transducers from 1 MHz to 20 MHz, for example. The one or more transducers or transducer array generates an electrical signal corrected for acoustic wavefront distortions so that it corresponds to an acoustic wave with planar wavefront. Particularly, the transducer array compensates for any distortions of the acoustic wavefront in order to achieve better resolution and image contrast. Information concerning the objects of interest in tissue is derived from the amplitude and frequency spectrum of the ultrasound waves detected by the transducers. Each transducer may be a piezoelectric transducer, such as single crystals of PZT or PMN), a capacitive micromachined ultrasonic transducer (CMUT) or an optical transducer, such as a laser beam deflection transducer, fiberoptic transducer or optical interferometer. Particularly, the central frequency and bandwidth of the ultrasonic transducer or transducers is preferably selected to provide as close as possible match to the broadband frequency spectra of acoustic signals generated in the object under investigation.

A fourth scanning subsystem is configured to position the collection and detection subsystems relative to the tissue of interest under examination with a high precision exceeding that of image resolution. A fifth electronic subsystem is comprises analog amplification, analog-to-digital conversion and digital signal processing components configured for signal acquisition and transmission thereof in the digital form. A sixth computer subsystem comprises software for signal processing, image reconstruction and image processing connected to a display showing images of the absorbed optical energy that can be converted into functional and molecular images.

Particularly provided is an optoacoustic imaging system and method of utilizing an off-axis parabolic an off-axis parabolic reflector made of suitable materials to perform an ideal conversion of a spherical ultrasound wave into a planar wave without losses in a wide range of apertures, for example, an off-axis parabolic reflector with an average numerical aperture greater than 0.4. To achieve the complete reflection of ultrasound from a surface of an off axis parabolic reflector, the angle of incidence a must exceed the critical angle of total internal reflection defined by a shear wave velocity in the material of the reflector in accordance with a Snell's law: sin α/C_{Lw}≧1/C_{sw}, where C_{Lw} and C_{sw} are the longitudinal and shear wave sound velocities propagating in a liquid coupling medium and the material of the reflector, respectively. If the angle of incidence is the critical angle, the ultrasound wavefront is significantly distorted. These wavefront distortions will degrade the performance, e.g., contrast and sensitivity, of such system and reduce its resolution. Off axis parabolic reflectors made of materials with high acoustic impedance value, such as brass or copper, will loose about 20% of ultrasound energy for angles of incidence exceeding the critical angle threshold. Due to their relatively high critical angle threshold of about 37 degrees, these materials are not suitable substrates for an off-axis parabolic reflector.

A relatively small number of materials with sufficiently high shear wave velocity can be used to manufacture the reflector with high numerical aperture. These include fused silica, selected types of glasses, for example, but not limited to Pyrex, selected types of steel, and aluminum alloys, such as, duralumin 17s, alloy 1100, alloy 6061). Note that aluminum (z=17 MRayls) and fused silica (z=12 MRayls) have relatively small acoustic impedance values compared to brass and copper (z=41, 44 MRayls). The most preferred material fused silica, which can be fabricated with diamond grinding and polishing. Most materials with high acoustic impedance that can be easily machined or cast, for example, copper, brass, lead, are not suitable as a substrate for reflector due to their relatively low shear wave velocities. Table 1 shows the properties of some materials which can be used for fabrication of acoustic parabolic preflector

TABLE 1

| | | | Critical Angle |
|---|---|---|---|
| Material | C_{sw}, m/s | C_{Lw}, m/s | deg. |
| Aluminum | ~3100 | 1540 | 29.8 |
| 1020 Steel | ~3240 | 1540 | 28.3 |
| Pyrex | ~3280 | 1540 | 28.0 |
| Fused silica | ~3750 | 1540 | 24.2 |

The present invention provides non-invasive imaging of the spatial, i.e., volumetric, distribution of the absorbed optical energy, which, after normalization, can be converted into distribution of the optical absorption coefficient in biological tissues in vivo up to several millimeters deep with a resolution provided by the diffraction limit of the parabolic acoustic reflector and bandwidth of the ultrawide band ultrasonic transducers or detectors. For example, the imaging systems and microcopes described herein are useful in a method for visualizing vasculature within a subject tissue, for example, human or other animal tissues, and for measuring hemoglobin concentration and its oxygen saturation. These imaging methods comprise functional or molecular imaging.

As described below, the invention provides a number of advantages and uses, however such advantages and uses are not limited by such description. Embodiments of the present invention are better illustrated with reference to the Figure(s), however, such reference is not meant to limit the present invention in any fashion. The embodiments and variations described in detail herein are to be interpreted by the appended claims and equivalents thereof.

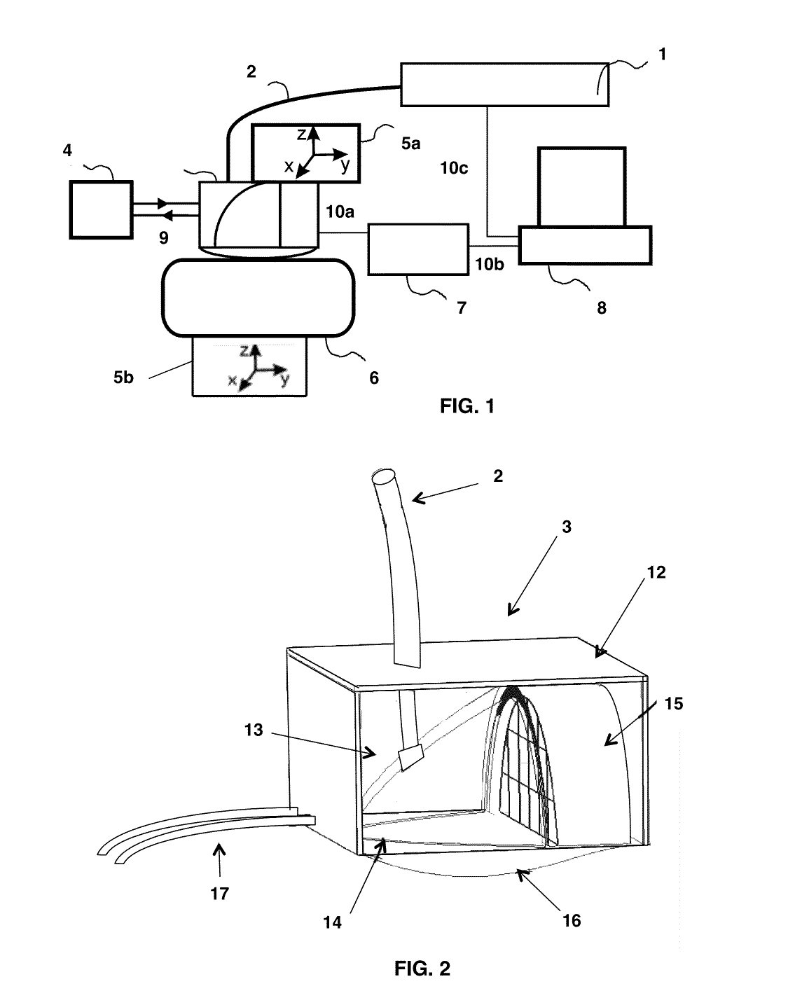

FIG. 1 illustrates the scanning high-resolution optoacoustic/ultrasonic imaging system. The imaging system comprises a short pulse laser 1 with tunable wavelength and a fiberoptic light delivery subsystem 2 in electronic communication with the laser and with an imaging module assembly 3. The components of the imaging module assembly are better depicted in FIG. 2, but generally comprise an acoustic parabolic reflector, an array of ultrawide band ultrasonic transducers, an optoacoustic coupling medium that fills the entire volume of the imaging module and is contained within by a thin, but strong, optically and acoustically transparent plastic, i.e., polymer, membrane. A pump 4 for the optoacoustic coupling medium is fluidly connected via input/output 9 to the imaging module assembly. A three-dimensional translational stage 5a is positioned in a movable relationship, along the X,Y,Z axes, with the imaging module assembly for scanning the imaging module over the stationary object 6 under examination. Alternatively, in some designs, the translational stage 5b can be positioned under the object 6 for translation of the object, where the imaging module assembly remains stationary. An electronics system 7 for analog signal amplification, conversion into digital form and signal and image processing is electronically linked at 10a,b to the imaging module assembly and to a computer 8. The computer also is electronically linked to the laser 1 at 10c. The computer comprises software 8a for the system control, data processing and image display 8b.

With continued reference to FIG. 1, FIG. 2 is a depiction of the main imaging module of a high resolution optoacoustic imaging system. The imaging module 3 comprises a housing or enclosure 12 through which the fiberoptic cable or bundle 2 passes. The fiberoptics deliver optical energy to the object or tissue under examination 6 through an optically and acoustically transparent coupling medium 13 preferably with speed of sound matched to the speed of sound in the object under examination.

The imaging module comprises an off-axis parabolic acoustic reflector 14 and a single-element or an array of ultrawide band ultrasonic transducers 15. A thin layer or membrane 16 is made of an optically and acoustically transparent material or plastic, such as polyethylene or similar material, is disposed within the housing to contain the coupling medium 13. Temperature of the liquid coupling medium may be adjusted to provide best matching of speed of sound in a coupling medium and an object under examination by using a circulation system with built in temperature controls. Liquid circulation outputs 17a,b are shown connected with the imaging module assembly.

Without limiting its scope, the present disclosure describes embodiments that provide an optoacoustic imaging system operating in backward mode that utilizes downward illumination to achieve better penetration of light into tissues. However, those skilled in the art must understand alternative geometries of light delivery are possible as permitted by geometry of an off-axis parabolic reflector, material of the reflector, and the object under investigation. Coupling medium may include, but are not limited to, water, alcohol, water/alcohol mixtures, water with dissolved salts, organics solvents, hydrogels or other materials.

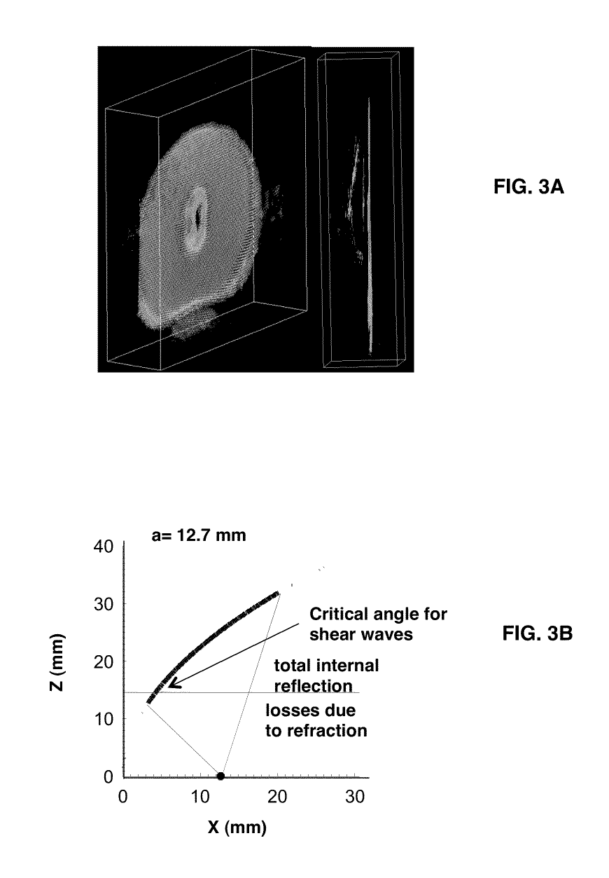

FIGS. 3A-3B depict renderings of an ultrasound wavefront. FIG. 3A shows a volumetric rendering of an ultrasound wavefront emitted by a point source in focus and reflected from an off-axis parabolic aluminum mirror, showing distortions of ultrasound wavefront in the lower part of the reflector. FIG. 3B shows the cross-section of the reflector, as well as the position of critical angle for shear waves in the material of the reflector, where presence of wavefront distortions is expected. Distortions in the central area arise due to the presence of a hole drilled for sample illumination.

FIGS. 4A-4B illustrate the off-axis parabolic reflector. By definition, a parabola is a set of points equidistant form focus and a line called directrix. FIG. 4A shows the example of a cross-section of an off-axis parabolic reflector depicted in the Embodiment 2, represented by a parabola

$$X = \frac{{Z}^{2}}{4 \times a} , a = 8 mm .$$

Point P₀ shows the focus of the reflector. Spherical wave emitted from focus will be converted into a plane wave by the off-axis parabolic reflector. It follows from the definition of the parabola that the distance d that the sound has to travel before it reaches the transducer is equal Rₓ+a. Angles α and β show the apertures of the reflector, which equal

$$sin α = \frac{a - {f}^{2} / 4 a}{a + {f}^{2} / 4 a}$$

$$sin β = \frac{{R}_{x} - a}{{R}_{x} + a} ..$$

For the parabolic reflector used in FIG. 2 the numerical aperture along X axis equals sin (α)=sin(β)=0.6. Angle of incidence of a spherical wavefront onto a surface of the reflector varies from it minimum value γ₁ at point Q to its maximum value γ₂ at point R, which can be parameterized as

$$γ ( x ) = 90 - arctan [ \frac{2 Z}{4 a} ] - arctan [ \frac{a - {Z}^{2} / 4 a}{Z} ]$$

FIG. 4B shows the change of γ across the surface of the reflector. Horizontal lines show critical angle for shear waves in selected materials.

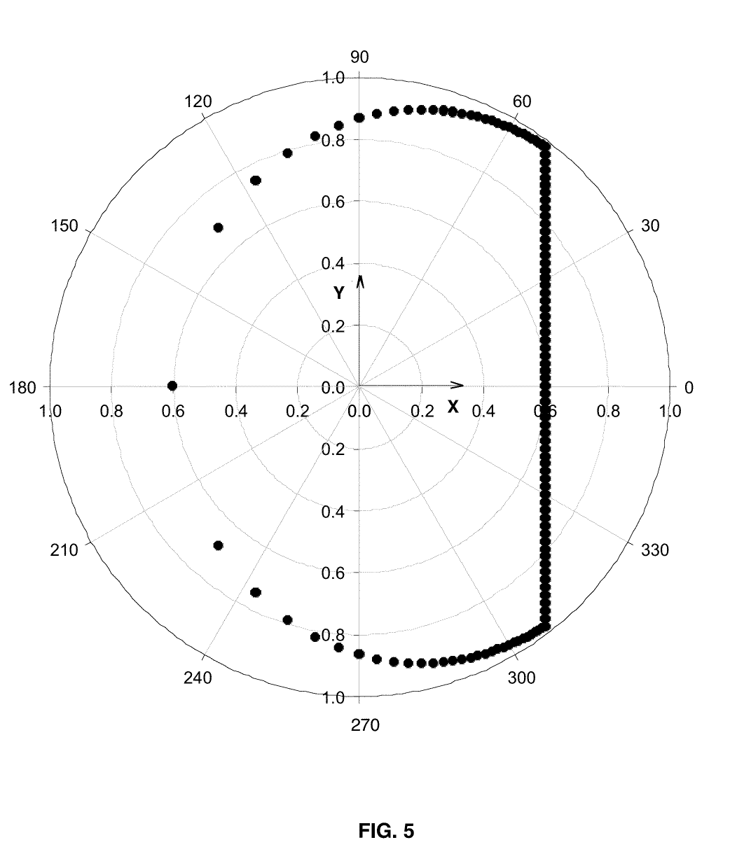

FIG. 5 is a polar plot depicting the variation in numerical aperture (NA) values of the off-axis parabolic reflector for different directions in the XY plane of the sample. The numerical aperture values along X and Y axis are 0.6 and 0.86, respectively.

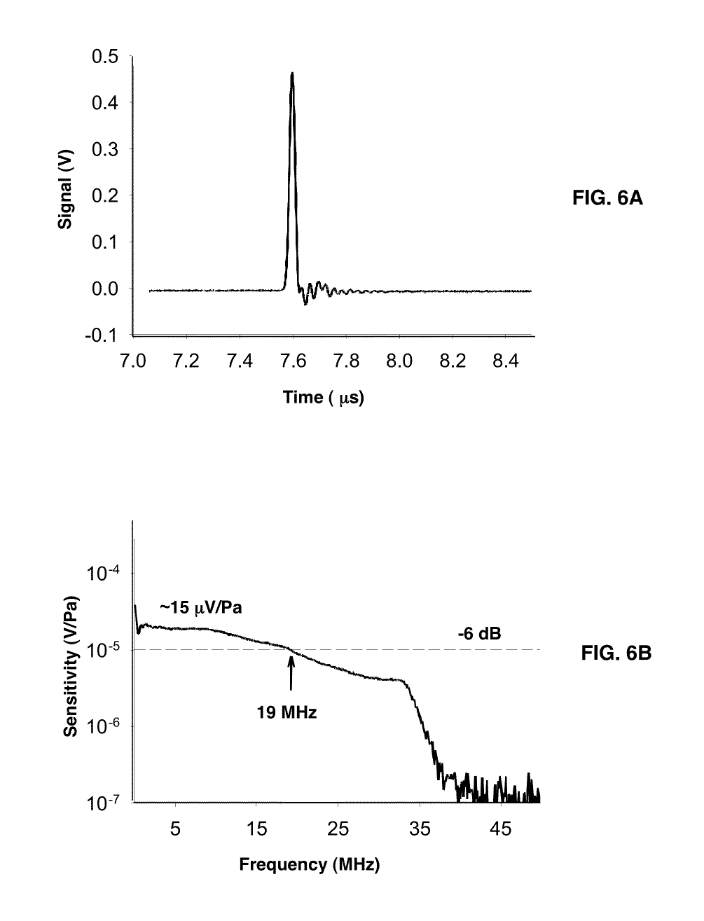

FIGS. 6A-6B illustrate the functioning of an ultrawide-band ultrasonic transducer comprising PVDF copolymer film. FIG. 6A shows impulse response and FIG. 6B shows the corresponding frequency spectrum of the ultrawide-band ultrasonic transducer. The transducer bandwidth of 19 MHz, and sensitivity of 15 μV/Pa was measured.

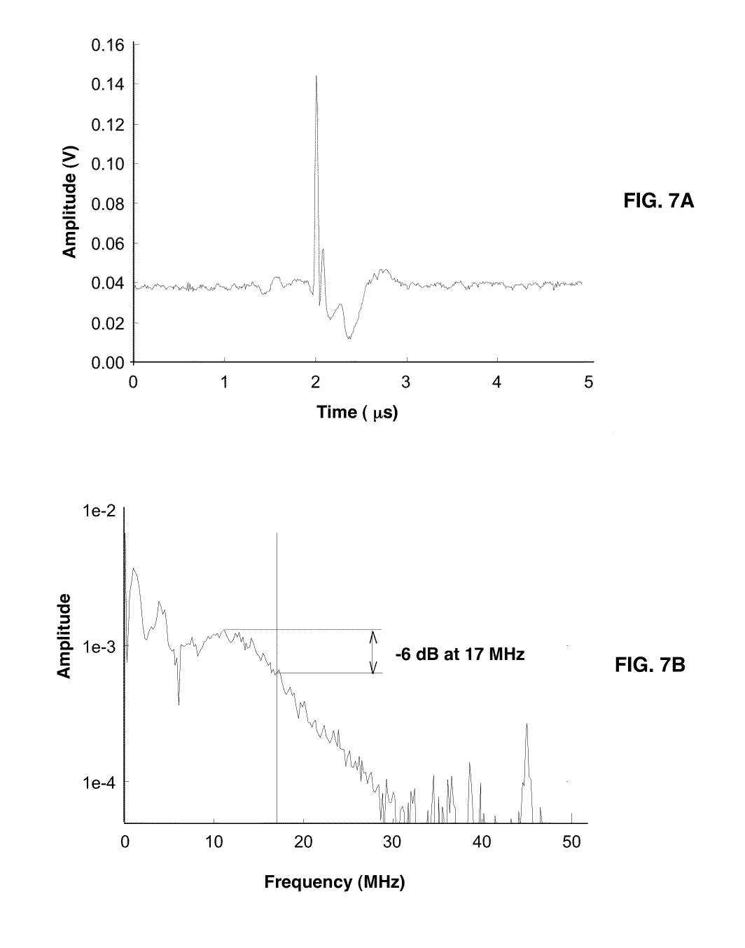

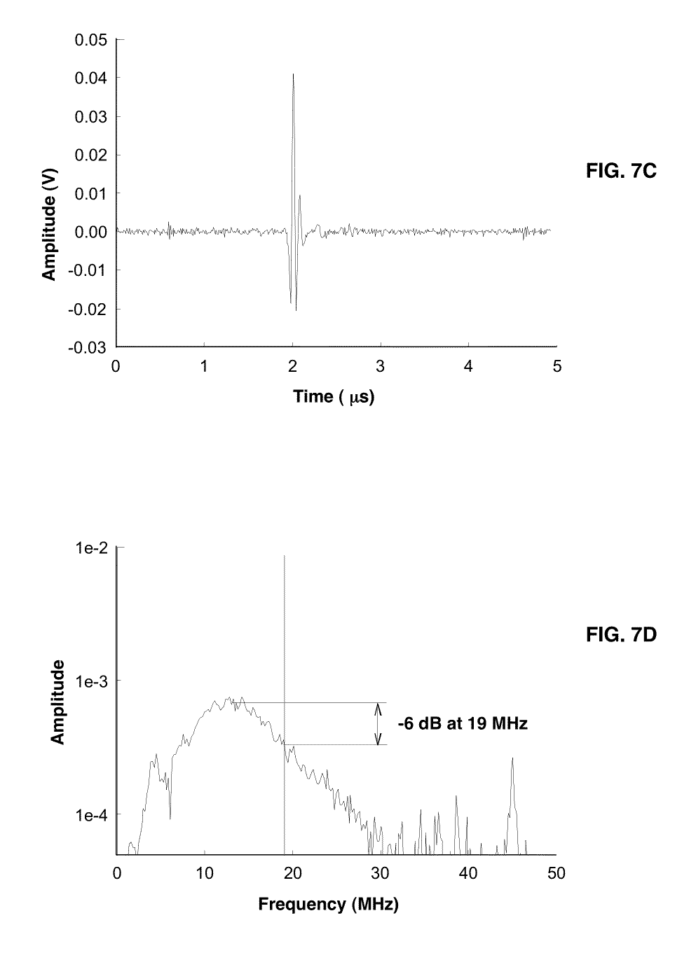

FIGS. 7A-7D are temporal profiles of impulse response signals and the corresponding Fast Fourier Transform spectra (FFT) of these signals from an optically absorbing microsphere with diameter of ˜35±3 m submerged in an optically scattering tissue phantom made of gelatin. The signals are recorded by the imaging system described herein. FIG. 7A is the raw signal and FIG. 7B is the corresponding FFT frequency spectrum, FIG. 7C is the signal processed with a 10 MHz high pass Butterworth filter and FIG. 7D is the corresponding FFT frequency spectrum.

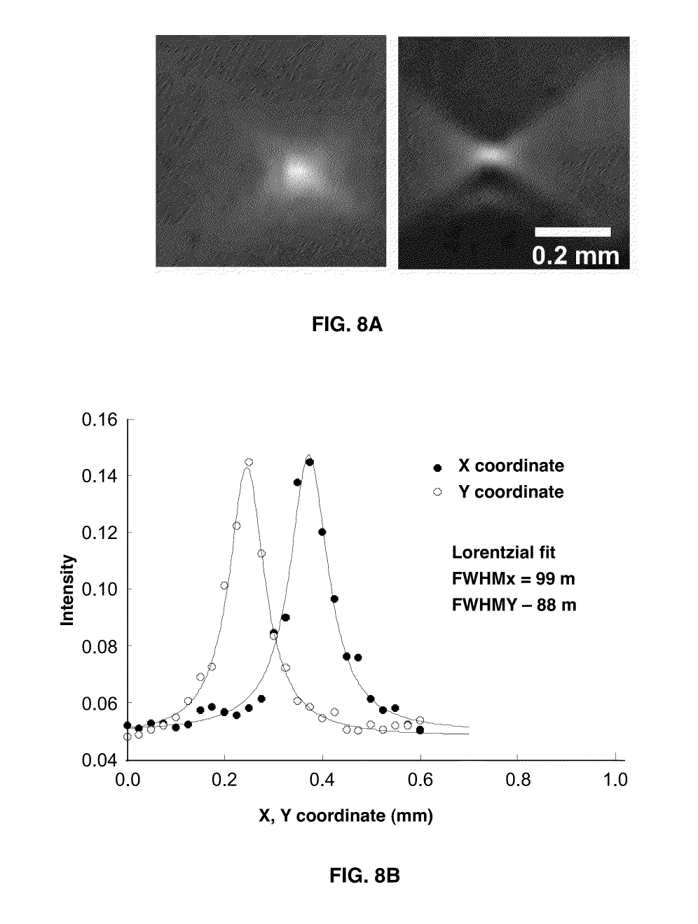

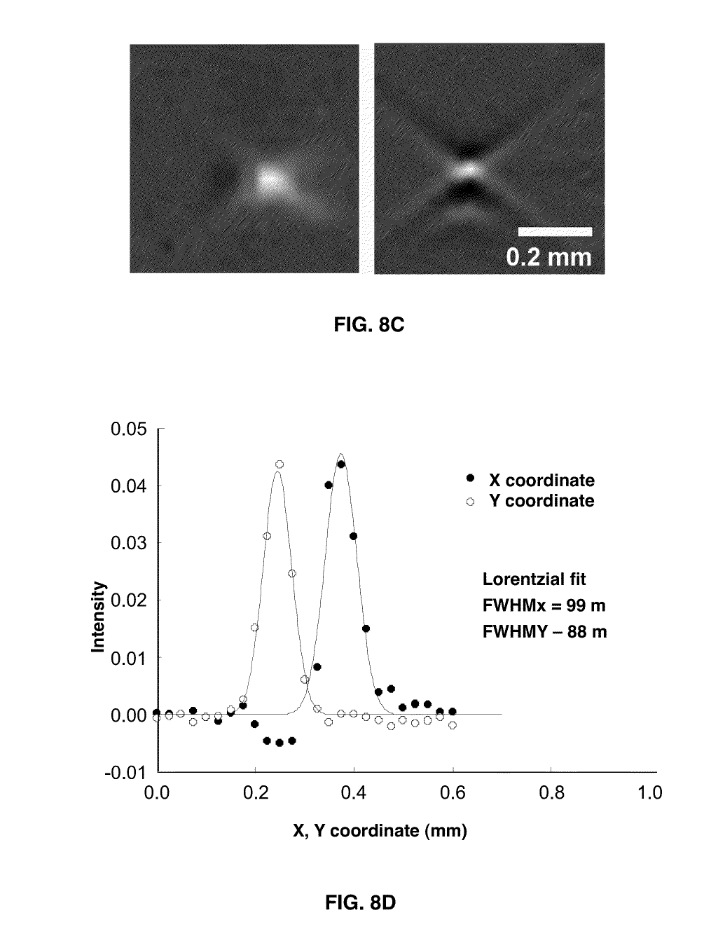

FIGS. 8A-8D are maximum intensity projection (MIP) images of an optically scattering phantom containing optically absorbing microspheres with diameter of ˜35±3 m. Images are of a single microsphere. Images were acquired using the pulsed laser emission at 762 nm with pulse duration of 12 ns. Images are presented at various levels of resolution along different axes. FIG. 8A is the reconstructed MIP image of the single microsphere and FIG. 8B is the cross-sections through this image along the X-axis (black dots) and the Y-axis (open circles) along with the corresponding Lorentzian fits. This image was reconstructed without any additional processing of the acquired transient signals. FIG. 8C is the reconstructed MIP image of the single microsphere after filtering the acquired signals with 10 MHz high-pass Butterworth filter and FIG. 8D is the corresponding cross-sections through the image along the X-axis (black dots) and the Y-axis (open circles) along with the corresponding Gaussian fits.

Note, that due to pulse duration of 12 ns and microsphere size of ˜35 μm are expected to reduce resolution of the image by ˜12-15%. Also note, that optoacoustic signals in the reconstructed images carried the central frequency of 10-12 MHz. Also note, that optoacoustic signals used in the image reconstruction had the central frequency of 10-12 MHz. One skilled in the art can project that imaging at 5-fold smaller acoustic wavelengths corresponding to a frequencies of ˜50 MHz will yield 5-fold better lateral resolution.

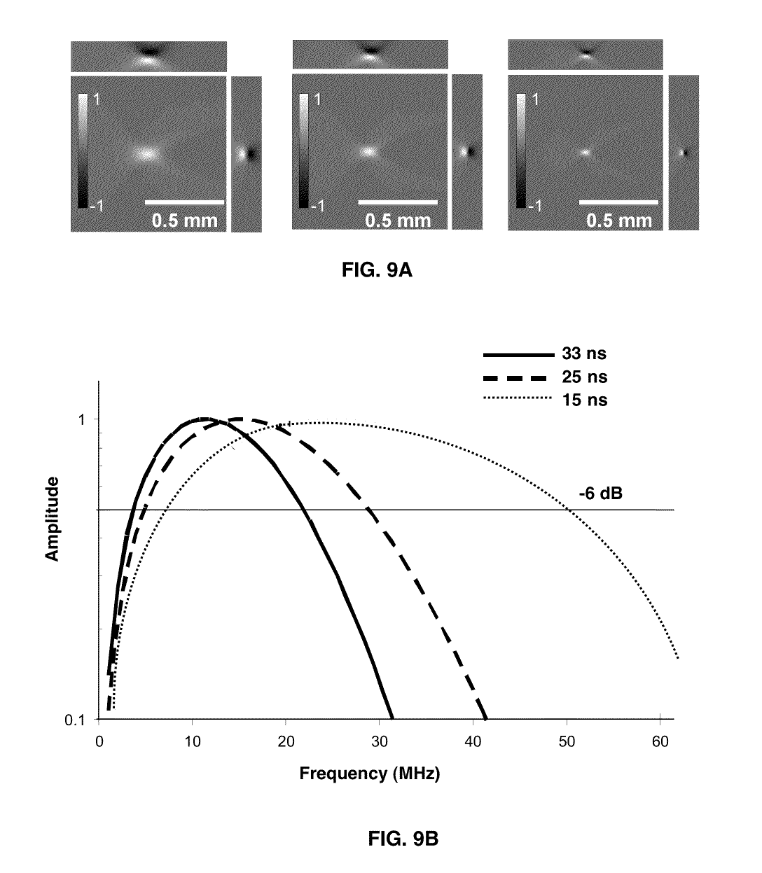

FIGS. 9A-9B depict optoacoustic images and the corresponding signals used for used for image reconstruction. FIG. 9A shows optoacoustic images of the diffraction limited point spread functions. FIG. 9B shows signals having temporal of a Gaussian derivative that were used for reconstructing the images. Gaussians profiles with duration at full width at half maximum (FWHM) of 33 ns, 25 ns, and 15 ns were selected for simulations. Speed of sound in the calculations is assumed to be equal to 1500 m/s. Table 2 shows Full Width at Half Maximum (FWHM) values of a Point Spread Function (PSF) measured at of a cross-sectional profile of the image brightness.

TABLE 2

| Gauss FWHM, | | | |
|---|---|---|---|
| ns | Lateral X, μm | Lateral Y, μm | Axial, μm |
| 33 | 123 | 72 | 100 |
| 25 | 85 | 53 | 75 |
| 15 | 48 | 31 | 45 |

The following references are relied on herein.
1. Zhang et al. J. Biomed. Opt. 17(2):020501-1-020501-4, (2012).
2. Dmitri et al. Proc. SPIE. 7899, Photons Plus Ultrasound: Imaging and Sensing 2011 78991D (Feb. 10, 2011) doi: 10.1117/12.877723
3. Dmitri et al. Proc. SPIE. 8223, Photons Plus Ultrasound: Imaging and Sensing 2012 82230Z (Feb. 9, 2012) doi: 10.1117/12.911015
4. Goss et al. Journal of the Acoustical Society of America 64(2)423-57, (1978).
5. Kino, G. S., Acoustic Waves: Devices, Imaging, and Analog Signal Processing, Prentice-Hall; (1987).
6. Oraevsky et al. “Time-Resolved Optoacoustic Imaging in Layered Biological Tissues”, in Advances in Optical Imaging and Photon Migration, Academic Press, p. 161-165 (1994).
7. Oraevsky, A. A. and Karabutov, A. A., “Optoacoustic Tomography” in Biomedical Photonics Handbook, CRC Press, p. 34-1-4 (2003).
8. Xu M. H. and Wang L. H. V., Review of Scientific Instruments 77(4):041101-1-6 (2006).
9. Savateeva et al., SPIE Proceedings 3916:55-66 (2000).
10 Maslov et al. Optics Letters 30(6):625-627 (2005).
11. Zhang et al. Nature Protocols 2(4):797-804 (2007).
12. Zhang et al. Applied Optics 47(4):561-577 (2008).
13. Laufer et al. Applied Optics 48(10), D299-D306 (2009).
14. De La Zerda et al. Nano Letters 10(6):2168-2172 (2010).
15. Song K. H. and Wang L. V., Journal of Biomedical Optics 12(6), 060503-1-3 (2007).
16. Fronheiser et al. SPIE Proceedings 6856, R0-R7 (2008).
17. Zhang et al. Nature Biotechnology 24(7):848-51 (2006).
18. Maslov et al. IEEE Transactions on Ultrasonics Ferroelectrics and Frequency Control 44(2), 380-385 (1997).
19. Zhang et al. Physics in Medicine and Biology 54(4):1035-1046 (2009).
20. Onda, “Tables of Acoustic Properties of Materials”, www.ondacorp.com/tecref_acoustictable.shtml.
21. Duck, F. A., Physical Properties of Tissues: a Comprehensive Reference Handbook, (1990).
22. Conjusteau et al. Review of Scientific Instruments 80(9):093608-1-6 (2009).
23. Hadiomioglu, B. and Quate, C. F., Appl. Phys. Lett. 43:1006 (1983).
24. Ryan et al. Proceedings of the IEEE Ultrasonics Symposium 1992, 2:1101-1105.
25. Tsyboulski et al. Proc. SPIE, 7899:78991D-1-78991D-8, (2011).
26. Tsyboulski et al. Proc. SPIE, 8223:82230Z-1-82230Z-6, (2012).
27. Nuster et al. J. Biomed. Opt. 17:030503-1-030503-3, (2012).

The present invention is well adapted to attain the ends and advantages mentioned as well as those that are inherent therein. The particular embodiments disclosed above are illustrative only, as the present invention may be modified and practiced in different but equivalent manners apparent to those skilled in the art having the benefit of the teachings herein. Furthermore, no limitations are intended to the details of construction or design herein shown, other than as described in the claims below. It is therefore evident that the particular illustrative embodiments disclosed above may be altered or modified and all such variations are considered within the scope and spirit of the present invention. Also, the terms in the claims have their plain, ordinary meaning unless otherwise explicitly and clearly defined by the patentee.

What is claimed is:

1. A scanning three dimensional optoacoustic imaging system, comprising:
a) an electromagnetic energy delivery subsystem having a source of electronmagnetic energy deliverable to an object of interest in a subject;
b) an ultrasound collection subsystem comprising an off-axis parabolic reflector disposed in a focusable relationship with a voxel of the object of interest that received the electromagnetic energy, said voxel generating detectable ultrasonic waves;
c) an ultrasound detection subsystem comprising at least one ultrawide-band ultrasonic transducer configured to convert the detectable ultrasonic waves to an electrical signal;
d) a scanning subsystem disposed in movable relationship to the ultrasound collection subsystem and ultrasound detection subsystem;
e) an electronic subsystem comprising analog amplification, analog-to-digital conversion and digital signal processing components configured for acquisition and digital conversion of the electrical signals; and
f) a computer comprising a memory and a processor tangibly storing software configured for signal processing, image processing and reconstruction, said computer in electronic communication with the electronic subsystem and the electromagnetic energy delivery subsystem.

2. The optoacoustic imaging system of claim 1, further comprising a housing subsystem that contains:
a) an enclosure enclosing the ultrasound collection subsystem and the ultrasound detection system;
b) a coupling medium filling the enclosure; and
c) a membrane that is transparent to both electromagneric radiation and ultrasound disposed within the enclosure and containing the coupling medium therein.

3. The optoacoustic imaging system of claim 1, further comprising a laser ultrasound imaging system for dual modality optoacoustic/laser ultrasound imaging, said laser ultrasound imaging system configured to operate with the subsystems 1b-1f and having:
a) an absorbing layer that generates a broadband ultrasound pulse upon illumination with an optical pulse, and
b) means for directing optical pulse onto the said absorbing layer.

4. The optoacoustic imaging system of claim 1, wherein the source of electromagnetic energy is a laser configured to pulse a specific wavelength of optical energy.

5. The optoacoustic imaging system of claim 1, wherein the source of electromagnetic energy is a physical source configured to pulse or to produce a modulated continuous wave of a wavelength in a range of about 100 nm to about 10 cm.

6. The optoacoustic imaging system of claim 1, wherein the numerical aperture of the off-axis parabolic reflector appears in a range of about 0.4 to about 0.99.

7. The optoacoustic imaging system of claim 1, wherein the ultrawide-band ultrasonic transducer has a bandwith in a range of about 1MHz to about 50 MHz.

8. The optoacoustic imaging system of claim 1, wherein the at least one ultrawide-band ultrasonic transducer comprises a transducer array.

9. The optoacoustic imaging system of claim 1, wherein the at least one transducer is a piezoelectric transducer, an optical transducer or a capacitive micromachined transducer.

10. The optoacoustic microscope of claim 1, wherein the object is either a tissue, or particles or molecules contained therein.

11. The optoacoustic imaging system of claim 1, wherein the computer is electronically connected to a means for displaying the reconstructed image.

12. A scanning optoacoustic microscope, comprising:
a source of a single or of multiple optical wavelengths absorbable by an object of interest;
an off-axis parabolic reflector having a high numerical aperture movably disposed in a focusing relationship onto a voxel within the object of interest;
an ultrawide band ultrasound transducer array disposed in a movable relationship with the off-axis parabolic reflector, said transducer array configured for conversion of ultrasonic waves to an electrical signal;
a scanning assembly disposed in a movable relationship to both the off-axis parabolic reflector and the transducer array; and
a computer system in electronic communication with the laser and with electrical signal acquisition and transmission components, said computer system comprising a memory, a processor and a display, tangibly storing software configured for signal processing and image reconstruction and processing.

13. The optoacoustic microscope of claim 12, further comprising:
a housing enclosing the off-axis parabolic reflector and the ultrawide-band ultrasonic transducer array;
an optoacoustic coupling medium filling the housing; and
an optically and acoustically transparent plastic polymer membrane disposed within the housing and containing the coupling medium therein.

14. The optoacoustic microscope of claim 12, further comprising a laser ultrasound imaging subsystem for dual modality imaging with the optoacoustic microscope, said laser ultrasound imaging system configured to operate with components of the optoacoustic microscope and having:
a) an absorbing layer that generates a broadband ultrasound pulse upon illumination with an optical pulse, and
b) means for directing optical pulse onto the said absorbing layer:

15. The optoacoustic microscope of claim 12, wherein the source of the single or multiple optical wavelengths comprises one or more lasers configured to produce pulses of optical energy at a selected wavelength or wavelengths.

16. The optoacoustic microscope of claim 12, wherein the frequency range of the detectable ultrasonic wavelengths appears in the range of about 1 MHz to about 50 MHz.

17. The optoacoustic microscope of claim 12, wherein the numerical aperture of an off-axis parabolic reflector is about 0.4 to about 0.99.

18. The optoacoustic microscope of claim 12, wherein the object is a tissue, or particles or molecules contained therein.

19. A scanning three-dimensional optoacoustic imaging method for collecting volumetric image data voxel-by-voxel in a subject, comprising the steps of:
a) scanning a tissue of interest in the subject with optical energy having a wavelength absorbed by one or more voxels within the tissue or a molecule therein;
b) collecting ultrasonic waves generated by each voxel within the tissue or molecule as each absorbs the optical energy via the off-axis parabolic reflector comprising the scanning optoacoustic microscope of claim 12;
c) detecting the collected ultrasonic waves with the ultrawide-band ultrasonic transducer array comprising the optoacoustic microscope and correcting wavefront distortions therein;
d) converting the corrected ultrasonic waves to electrical signals which are processed to a digital format and transmitted to the computer system for reconstruction as a volumetric image of the optical energy absorbed by each of the voxels.

20. The optoacoustic imaging method of claim 19, further comprising converting the volumetric image of the absorbed optical energy into functional and molecular images of the tissue or molecule therein.

21. The optoacoustic imaging method of claims 19, further comprising combining the optoacoustic microscope in dual modality with a laser ultrasound imaging system to acquire and co-register anatomical images of tissue morphology with the functional and molecular images.

22. The optoacoustic imaging method of claim 19, wherein the scanning step comprises:
delivering the optical energy through an optoacoustic coupling medium contained within an optically and acoustically transparent membrane.

23. The optoacoustic imaging method of claim 22, wherein the collecting step comprises:
positioning the off-axis parabolic reflector confocally with the optical energy on the tissue of interest such that each voxel in the tissue of interest is located at the common focal point on the same axis.

24. The optoacoustic imaging method of claim 22, wherein the off-axis parabolic reflector converts spherical acoustic waves generated by each voxel that are incident at an ultrawide range of angles into a planar acoustic wave propagated as ultrasound.

25. The optoacoustic imaging method of claim 22, wherein the corrected electrical signal corresponds to an acoustic planar wavefront.

26. The method of claim 19, wherein the tissue is vascular tissue and the molecule is one or both of hemoglobin or oxygen.
